(19) United States
(12) Patent Application Publication
(10) Pub. No.: US 2009/0304587 A1
(43) Pub. Date: Dec. 10, 2009

(54) BIODEGRADABLE COMPOSITE FOR INTERNAL LOCAL RADIOTHERAPY

(75) Inventors: Abraham Rubinstein, Jerusalem (IL); Abdel Kareem Azab, Ara (IL); Boris Orkin, Jerusalem (IL); Aviram Nissan, Jerusalem (IL); Susan Haupt, Zur Hadassa (IL); Morris Srebnki, Mevasseret Zion (IL); Raphael Udassin, Jerusalem (IL)

Correspondence Address: BROOKS KUSHMAN P.C., 1000 TOWN CENTER, TWENTY-SECOND FLOOR, SOUTHFIELD, MI, 48075, US

(73) Assignees: Yissum Research Development Company Of The Hebrew University of Jerusalem, Jerusalem (IL); Hadasit Medical Research Services & Development Limited, Jerusalem (IL)

(21) Appl. No.: 12/092,350

(22) PCT Filed: Nov. 1, 2006
(86) PCT No.: PCT/IL06/01259

§ 371 Date: Jan. 5, 2009

Related U.S. Application Data
(60) Provisional application No. 60/731,872, filed on Nov. 1, 2005.

(51) Int. Cl. A61K 51/04

(52) U.S. Cl. 424/1.73; 424/1.65; 424/1.85

(57) ABSTRACT

The present invention discloses composites which generally comprise a polymeric matrix and a hydrophobic organic compound which is associated with a radioisotope. The composites are biocompatible and biodegradable hydrogels suitable for use in internal local radiation therapy

### FIELD OF THE INVENTION

The present invention generally relates to composites for internal local radiotherapy.

### BACKGROUND OF THE INVENTION

Radiotherapy is the medical use of ionizing radiation for the treatment of a variety of disorders, among which cancer treatment is the most widespread. Radiotherapy is used for either curative or adjuvant treatments of cancer. However, it is often used palliativly to accomplish local control over metastatic spread and it is most common to blend radiotherapy with surgery, chemotherapy, hormone therapy and combination of thereof.

Because radiotherapy is applied to the gross tumor and marginal normal tissues (and sometimes to neighboring draining lymph nodes), healthy tissues are often damaged by the high energy of the external beam used (external beam therapy). One way to reduce the injury is to use shaped radiation beams, from different angles (at a distance of 50 cm to several meters) to intersect the tumour, a tactic that provides a larger radiation dose than in the surrounding, healthy tissues.

The alternative approach is the use of brachytherapy. In brachytherapy (“short distance therapy”) the radioactive source (metal seeds or ribbons) is implanted either within (interstitial implants) or in close proximity (intra-cavitary implant) to the tissues of interest or in contact with the tissue at risk. In accord with the tumor type brachytherapy is categorized as (a) mould brachytherapy for the treatment of superficial tumors (skin); (b) surface brachytherapy, where a tinny applicator (hollow thin silver casting, containing a radioactive source) is placed on the ill organ; (c) interstitial brachytherapy, where radioactive sources (metal needles) are inserted into the tissue (e.g. prostate); (d) intracavitary brachytherapy, in which the sources is implanted inside a pre-existing body cavity and (e) intravascular brachytherapy, where a loaded catheter is placed inside the vasculature (in-stent restenosis).

Permanent implants using radioactive “seeds” containing various radiation sources such as ¹²⁵I have been employed. ¹³⁷Cs, ¹⁹²Ir, and ¹⁰³Pd sources have been employed in temporary implants. The use of ¹³³Xe and ¹³¹Xe has also been suggested.

European Patent No. 0979656 discloses a radioactive composition for use in the therapy of neoplastic and non-neoplastic diseases by means of application of radioactive sources in direct contact with the tumor tissues or within them, and for use as an aid in radio-guided surgery. The composition contains radioisotopes immobilized on biocompatible or bioabsorbable solid particles, incorporated in a biocompatible and bioabsorbable matrix consisting of a hypotonic gel containing 2 to 30% w/w polyvinylpyrrolidone and 0.01 to 2% w/w of agar-agar or agarose in water.

International Patent Publication No. WO 97/19706 discloses therapeutic sources for use in the practice of brachytherapy. They are made of radioactive powder of ¹⁰³Pd, ¹⁹²Ir, ⁹⁰Yt, ³²P or ¹⁹⁸Ag dispersed in a biocompatible polymeric matrix. The polymeric matrix is desirably manufactured with pre-selected flexibility suitable to its intended use, e.g. in the form of a rod, hollow rod, suture, film, sheet, or microspheroidal particles. The radioactive composites generate a substantially uniform radiation field in all directions. The radiation dose is assembled by from the radioactive composite during the medical procedure to emit a desired amount of therapeutic radiation. Optionally, the polymer is selected to dissolve or degrade in the body at a predetermined rate, depending upon the half-life of the radioisotope used in the therapeutic source.

International Patent Publication No. WO 95/16463 discloses the use of therapeutic radioactive compositions comprising radioactive elements such as ¹⁶⁶Ho, ¹⁵³Sm, ¹⁰⁵Rh, ¹⁷⁷Lu, ¹⁹²In, ¹⁶⁵Dy, ⁹⁰Y, ¹⁴⁰La, ¹⁵⁹Gd, ¹⁷⁵Yb, ¹⁸⁶Re, ¹⁸⁸Re and ⁴⁷Sc, adsorbed onto cellulose ether or a derivative thereof in treating rheumatoid arthritis.

### SUMMARY OF THE INVENTION

While brachytherapy has proven safer for adjacent healthy tissues, complicated placement and removal procedures associated with its application seriously constrained implementation of this therapy regimen. The use of biodegradable devices which are pre-loaded with radioisotopes, thus eliminating the need for their post-treatment extraction, was envisioned by the inventors of the present application as a mean to remove much of the complexity and discomfort associated with brachytherapy.

The inventors of the present invention have developed a novel radioactive and biodegradable composite which was found suitable for local internal radiation therapy, such as branchytherapy. This composite comprises a polymeric matrix embedded with a hydrophobic organic compound, such as a lipid, associated with at least one beta- or gamma-emitting radio-isotope.

The inventors have found that two general sub-groups of such a composite were suitable for internal radiation therapy:
(i) a composite of a polymeric matrix and an hydrophobic organic compound, such as a lipid, covalently bonded to a halogen radioisotope; and
(ii) a composite of a polymeric matrix and an hydrophobic organic compound, such as a lipid, e.g. cholesterol chemically associated, preferably via coordinative association (not via covalent bonding) with at least one beta- or gamma-emitting radioisotope such as ¹²⁴I, ¹²⁵I, ¹³¹I, ⁹⁰Y, ¹⁶⁶Ho, ¹⁸⁶Re, ¹⁸⁸Re, ⁹⁰Sr, ²²⁶Ra, ¹³⁷Cs, ⁶⁰Co, ¹⁹²Ir, ¹⁰³Pd, ¹⁹⁸Au and ¹⁰⁶Ru.

The composites of the invention posses the following properties:

(i) they are fully biocompatible;

(ii) they are fully biodegradable, thus eliminating the need for post-treatment extraction;

(iii) they are loadable with therapeutic doses of radioactive compounds without substantially affecting the biocompatibility and/or biodegradability of the matrix;

(iv) the composites' degradation rate and residence time in the body are controllable and engineerable according to the therapeutic needs;

(v) they are useful in the treatment of diseases or disorders such as neoplasms with minimal effect to neighboring tissues and/or organs;

(vi) they may be used in combination therapies;

(vii) apart from the radioactive compound the composites may be loaded with other agents such as antineoplastic agents; and

(viii) the release of the radioactive compound into neighboring healthy tissue is substantially undetectable.

Thus, in a first aspect of the present invention, there is provided a composite of a polymeric matrix embedded with at least one hydrophobic organic compound associated with at least one radioactive atom, wherein said at least one hydrophobic organic compound associated with said at least one radioactive atom is substantially non-leachable from said matrix.

As used herein, the term “composite” refers to the product produced by combining the polymeric matrix and the organic compound employed in the invention. The embodiment of the hydrophobic organic compound in the matrix is preferably continuous and homogenous. As used herein, the term “embedded” or any lingual variation thereof contemplates any manner by which the radioactive compound is incorporated into the polymeric matrix, including for example: attachment to a monomer of the polymer and having the radioactive compound a part of the polymerization; distribution of the radioactive compound throughout the polymeric matrix; entrapment of the radioactive compound in voids within said matrix; random dispersion of the radioactive compound within the polymeric matrix; encapsulation inside the polymeric matrix, etc. The degree and uniformity of the embedment may also be a result of chemical and/or physical interactions between the matrix and the radioactive compound. Such interactions may be acid-base, hydrophibicity-hydrophilicity, ionic interactions, complexation, chelation, etc.

The term “polymeric matrix” refers to a biocompatible and biodegradable polymer, preferably to an organic polymer, in which the radioactive organic compound is embedded. Non-limiting examples of such polymers are polyurethane, polypropylene, polypropylene terephthalate, polyphenylene oxide, polyphenylsulfone, polyether sulfone, phenyletheretherketone, polyetherimide, silicone and liquid crystal polymer. Non-limiting examples of biocompatible and biodegradable polymers are polyglycaprone, polyglactin and polydioanone.

The polymeric matrix employed may be formed, as will be discussed hereinbelow, from the monomers of the polymer (by polymerization), from the polymer itself under appropriate conditions (such as those leading to hydrogelation) or in the presence of other agents such as curing or crosslinking agents which may be necessary in order to achieve the formation of a desired matrix.

In some embodiments, the polymer is a curable or crosslinkable polymer. The term “crosslinking”, as may be known to a person skilled in the art, refers to the reaction between a polymer and a second chemical entity which may be another polymer or a chemical compound to which the polymer is reactive. The crosslinking may for example be achieved by employing appropriate chemical conditions, as may be known to a person skilled in the art and demonstrated in the Examples. For example, the crosslinking may be of a polymer having repeating units which bare at least one amino group (such as in the case of chitosan) per unit, capable of crosslinking with a dialdehyde-containing compound. The crosslinking may also, for example, be affected by the addition of multivalent cations such as calcium.

The polymer consisting the matrix, in a most preferred embodiment of the invention, is a polysaccharide. The polysaccharide may have the same repeating monosaccharide units (such as in the case of cellulose) or different repeating units (such as in the case of alginic acid), may be natural, synthetic or semisynthetic (modified), branched or linear.

The polysaccharide may be employed in its salt form, namely charged. Typically, the salts are of the polysaccharide and cations such as calcium, magnesium, barium, aluminum, ferrous and ferric, and others.

Non-limiting examples of polysaccharides are alginic acid, amylopectin, amylose, arabinoxylan, cellulose, chitin, chitosan, chondroitin, galactoglucomannan, glucomannan, glycogen, guar gum, heparin, hyaluronic acid, inulin, pectin, and xyloglucan.

Non-limiting examples of crosslinking agents are glutaraldehyde, diaminododecane, and divinyl glycole. Crosslinking may also be achieved between two different polysaccarides.

In one embodiment, the polysaccharide is a curable or crosslinkable polysaccharide having monosaccharides which have at least one amino group (such as in the case of chitosan), which can be crosslinked with dialdehyde-containing crosslinking compounds (such as glutaraldehyde).

The term “hydrophobic organic compound” refers to an organic compound, i.e. having a carbon basis, which is substantially insoluble in water and which may be fully synthetic, partially synthetic or natural. The organic compound is additionally one which is biocompatible and which does not exert any additional toxic effect, apart from the effect exerted by the radiation, on the tissue or organ in which the composite is implanted. Within the context of the present invention, the hydrophibicity of the organic compound should substantially remain the same regardless of the type of association between the organic compound and the radioactive atom. The association between the organic compound and the radioactive atom may depend on the nature of each of the partners.

In some embodiments, the association is via at least one chemical bond, namely the two may be held together via bonding such as covalent, ionic, hydrogen, van der waals, coordination, etc. Preferably, in such embodiments, the association is of a covalent or coordinative nature.

In other embodiments, the association between the hydrophobic organic compound and the radioactive atom is physical, namely the radioactive compound may be entrapped or encapsulated such as in the case of micellar systems and inclusion complexes, e.g. liposomes, dendrimers, etc.

Preferably, the hydrophobic organic compound employed in the composite of the invention is selected from lipids such as cholesterol and norcholesterol, fats such as triglycerides, hydrocarbons of various chain lengths, and others.

The term “radioactive compound” refers herein to the hydrophobic organic compound when associated with at least one radioactive atom. The “radioactive atom” is preferably selected from: (a) gamma emitting radio-isotopes; (b) beta emitting radio-isotopes; or (c) a combination of a gamma emitting radio-isotope and a beta emitting radio-isotope.

In one embodiment, the radioactive atom is selected from radioactive halogens. In this case, the radioactive halogens (i.e. Br, Cl, I and F) are chemically bonded to the organic compound via covalent bonding.

The hydrophobic organic compound associated with the radioactive atom is said of being “substantially non-leachable from the matrix”. This means that regardless of the type of association between the organic compound and the radioactive atom (e.g. covalent, ionic, etc), the radioactive atom does not dissociate from the hydrophobic organic compound. Preferably, the radioactive compound (as defined above) as a whole does not dissociate from the matrix in which it is entrapped until sufficient disintegration of the matrix allows such dissociation. As discussed above, the radioactive compound substantially does not leach from the matrix. However, with the dissociation of the matrix, some leaching may occur. Preferably, the dissociation from the disintegrated matrix occurs after the radiation has substantially decayed.

In a preferred embodiment of the invention, there is provided a composite of a polymeric matrix, preferably a polysaccharide matrix, more preferably a hydrogel thereof, embedded with at least one hydrophobic organic compound, covalently bonded to at least one radioactive halogen, wherein said organic compound covalently bonded to said radioactive halogen is substantially non-leachable from said matrix. Preferably, the at least one radioactive halogen is selected from radioisotopes of iodine (such as ¹²⁴I, ¹²⁵I, ¹³¹I) and fluorine (such as ¹⁸F). Preferably the radioactive halogen is iodine with the most preferred isotope being ¹³¹I.

In another embodiment of the invention, the radioactive atom is selected from beta- and/or gamma-emitting radioisotopes such as ¹²⁴I, ¹²⁵I, ¹²⁷I, ¹³¹I, ¹⁸F, ⁹⁰Y, ¹⁶⁶Ho, ¹⁸⁶Re, ¹⁸⁸Re, ⁹⁰Sr, ²²⁶Ra, ¹³⁷CS, ⁶⁰Co, ¹⁹²Ir, ¹⁰³Pd, ¹⁹⁸Au, ⁹⁹Tc, ²⁰¹Th, ⁶⁷Ga, ¹¹¹In and ¹⁰⁶Ru and the organic compound with which said radioactive atom is associated is selected amongst lipids, fats, and hydrocarbons.

Preferably, said lipid is one of cholesterol and norcholeterol.

Thus, in another preferred embodiment of the invention, there is provided a composite of a polymeric matrix, preferably a polysaccharide, more preferably a hydrogel thereof, embedded with at least one lipid associated with at least one radioactive atom selected from beta- and/or gamma-emitting radioisotopes, wherein the association between said at least one radioactive atom and at least one organic compound is via an association selected from ionic bonding, coordination bonding, and intermolecular bonding. Preferably, the association is via coordinative bonding. The beta- or gamma-emitting radioisotope may for example be selected from ¹²⁴I, ¹²⁵I, ¹³¹I, ⁹⁰Y, ¹⁶⁶Ho, ¹⁸⁶Re, ¹⁸⁸Re, ⁹⁰Sr, ²²⁶Ra, ¹³⁷Cs, ⁶⁰Co, ¹⁹²Ir, ¹⁰³Pd, ¹⁹⁸Au, ⁹⁹Tc and ¹⁰⁶Ru.

As used herein, the term “association” or any lingual variation thereof, in the context of such an expression as “association between the organic compound and the radioactive atom” refers to the chemical or physical force which holds the two entities together. Such force may be any type of chemical or physical bonding interaction known to a person skilled in the art. Non-limiting examples of such association interactions are ionic bonding, covalent bonding, coordination bonding, complexation, hydrogen bonding, van der Waals bonding, hydrophobicity-hydrophilicity interactions, etc. In some preferred embodiments, the association is via covalent bonding. In other preferred embodiments, the association is via coordinative bonding.

It should be understood to a person skilled in the art that in some cases the associative interactions between two atoms or two chemical entities may involve more than one type of chemical and/or physical interactions.

The present invention thus provides composites of at least one polysaccharide matrix being preferably a hydrogel and an organic compound covalently bonded to at least one radioactive halogen; or with a lipid associated with at least one beta- or gamma-emitting radioactive atom.

In one embodiment, the polymeric matrix is embedded with a single radioactive compound. In another embodiment, the matrix is embedded with two or more different radioactive compounds, which may be of the same organic backbone bonded to different radioactive atoms or isotopes, or may be of different organic structures. For example, in one case the matrix may be embedded with a beta-emitting-cholesterol and gamma-emitting-cholesterol, and in another case be embedded with beta-emmiting-cholesterol and beta-emmiting-norcholesterol.

The composite of the invention may be used to treat a specific localized area (locoregion) of the body of the patient. The composite is fabricated so that it retains the radioactive organic compound for a pre-defined period of time without the radioactive compound substantially leaking or disassociating from the polymeric matrix in which it is embedded.

The polymeric matrix embedding the radioactive organic compound is biocompatible. The terms “biocompatible”, “biocompatibility” or any lingual variation thereof, when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host.

In certain embodiments of the present invention, biodegradation generally involves degradation of the polymer in a tissue or a body fluid, e.g., into its monomeric subunits, which may be known to be effectively non-toxic. Intermediate oligomeric products resulting from such degradation may have different toxicological properties, or biodegradation may involve oxidation or other biochemical reactions that generate molecules other than monomeric subunits of the polymer. Consequently, toxicology of a biodegradable polymer intended for in vivo use, such as implantation or injection into a patient, may be determined after one or more toxicity analyses.

It is not necessary that any subject composite have a purity of 100% to be deemed biocompatible; indeed, it is only necessary that the subject composites be biocompatible as set forth above. Hence, the composite of the invention may comprise polymers which are only 99%, 98%, 97%, 96%, 95%, 90%, 85%, 80%, 75% or even less biocompatible, e.g., including polymers and other materials and excipients described herein, and still be biocompatible.

To determine whether a polymer or other material is biocompatible, it may be necessary to conduct a toxicity analysis. Such assays are well known in the art, and are exemplified hereinbelow. In addition, polymers and composites of the present invention may also be evaluated by well-known in vivo tests, such as subcutaneous implantations in rats to confirm that they do not cause significant levels of irritation or inflammation at the subcutaneous implantation sites.

In the preferred embodiments, the polymeric matrix is adapted to be degraded and/or absorbed by the body. In such cases, the polymer is eliminated by the body over time, and the dissolution time is preferably chosen to be sufficiently greater than the radioactive half-life of the radioactive material, insuring that the remaining radioactivity does not poses any hazard to body tissues as it migrates from the treatment volume or site. As such, the polymeric matrix is based on biocompatible materials such as the polysaccharides mentioned hereinbefore, which are biodegradable and evoke no toxic response when released into the body.

The term biodegradable is art-recognized, and includes polymers, composites and formulations comprising thereof, such as those described herein, that are intended to degrade during in vivo use, such as implantation. In general, degradation attributable to biodegradability involves the degradation of a biodegradable polymer into its component subunits, or digestion, e.g., by a biochemical process carried out for example by enzymes, of the polymer into smaller, non-polymeric subunits.

Two different types of biodegradation may generally be identified. For example, one type of biodegradation may involve cleavage of bonds in the polymer matrix. In such biodegradation, monomers and oligomers typically result, and even more typically, such biodegradation occurs by cleavage of a bond connecting one or more of subunits of a polymer. In contrast, another type of biodegradation may involve cleavage of a bond internal to side chain or that connects a side chain to the polymer backbone. As used herein, biodegradation encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant (for example porosity), shape and size, and the mode and location of implantation. For example, the greater the molecular weight, the higher the degree of crystallinity, and/or the greater the biostability, the slower the biodegradation.

As stated herein, the composite of the invention is preferably a hydrogel of a polysaccharide. The term “hydrogel” is art-recognized and typically refers to a colloidal system with at least two phases, in which a dispersed phase (being in this case the polymer or polysaccharide) coexists with a continuous phase (being typically water) to form a continuous three-dimensional network which is generally viscous and jellylike.

The hydrogel may be prepared by a number of methods known in the art. One such exemplary method involves the admixing of a suitable polymer or a polysaccharide and the radioactive hydrophobic organic compound in pure water or in an aqueous solution containing e.g. acid. The hydrogelation may be spontaneous or may be achieved by e.g. heating the solution to a specific temperature or adjusting the pH.

Additionally, in order to achieve specific or improved physical matrix, a crosslinking agent may be added.

In order to achieve controlled or predetermined biodegradability, the hydrogel may be further treated, e.g. by washing or incubation, in appropriate conditions as discussed hereinbelow.

The composite of the invention may also be fabricated as a therapeutic source. As used herein, the term “therapeutic source” refers to a device that can be fabricated from the composite of the invention. This source may take on any structure or shape as may be determined suitable by the medical practitioner. The source may be any piece of the composite, or a well structured source such as in the shape of a cylindrical rod, a hallow rod, a suture, a mesh, a film, a sheet or spheroidal. While preferably the composite of the invention is biodegradable and requires no post-treatment extraction, the therapeutic source, as defined herein, may be designed and used as an extractable temporary implant or as a permanent implant.

Preferably, the composite or source fabricated therefrom or a composition comprising thereof is suitable for internal local radiation therapies such as brachytherapy, as well as for intracavity, interstitial, intraluminal and intravascular radiation therapy and/or for injection directly into a tumor.

The composite of the invention may also be contained within a second layer of polymeric material. This type of second polymeric material is typically not radioactive. The second polymeric material may in other cases be the core around which the composite of the invention is fabricated.

In certain embodiments, other materials may be dispersed in the polymer matrix. Such materials may for example be antineoplastic agents used for combination therapy of the same neoplasm or a different one, to alter the physical and chemical properties of the resulting polymer, including for example, the release profile of the resulting polymer matrix, etc. Examples of such materials include active ingredients, biocompatible plasticizers, delivery agents, fillers and the like.

The composite may also be suitable for the manufacture of or use as a radioactive medical implant for placement at a surgical site or a body cavity, i.e. any space or void within the body or in one of its organs, which may be normal or pathological. The medical implant may also serve as a supportive structure for preventing tissue collapse and for reducing tissue deformity.

In another aspect of the present invention, there is provided a method for the preparation of a composite of the invention, said method comprising:
(a) admixing at least one polymer and at least one hydrophobic organic compound associated with at least one radioactive atom in water or an aqueous solution; and
(b) affecting gelation or hydrogelation, thereby obtaining a composite of a polymeric matrix embedded with at least one hydrophobic organic compound associated with at least one radioactive atom, said at least one hydrophobic organic compound associated with said at least one radioactive atom being substantially non-leachable from said matrix.

In some embodiments, the method may utilize a pre-formed polymer or polysaccharide. In other embodiments, the method may further comprise the step of polymerization. In these cases, the method may involve the admixing of monomers or short oligomers or pre-polymers with the radioactive hydrophobic organic compound in a suitable media which would allow in situ polymerization and gelation or hydrogelation, thereby affording a composite of the invention.

In other embodiments, the polymer or polysaccharide employed may require curing or crosslinking. Such curing or crosslinking may be chemical, e.g. require the presence of a crosslinking agent, or physical, e.g. radiation curing. One such example of crosslinkable polysaccharide is chitosan which may be crosslinked in the presence of dialdehyde-containing agents.

The method may further also comprise the step of washing or incubating the hydrogel obtained in a suitable media. The degree of polymerization (cross-linking) of the gel and post-curing processes, such as washing and incubation in various media, may be used to control the viscosity of the hydrogel obtained, its elastic/plastic properties, and its in vivo degradation profile. Specifically, a composite having predetermined (controllable) degradation properties may be prepared by incubating the composite of the invention in media with different properties selected from buffer capacity, pH, ionic strength and osmolarity, each capable of modifying the biodegradation properties of the polymer.

For example, when the composite of the invention is subjected to rinsing with a phosphate buffer solution a slow degrading composite (SDC) is obtained; whereas, when water is used as the rinsing medium, a fast degrading composite (FDC) is obtained.

Thus, the present invention further provides a method for controlling the biodegradability of a composite of the invention, said method comprising washing or incubating a composite as disclosed herein with a suitable media. In one case, the suitable media is water, thereby obtaining a fast degrading composite (FDC). In another case, the media is a phosphate buffer solution, thereby obtaining a slow degrading composite (SDC). Preferably, the FDCs of the invention are prepared such that they biodegrade during about a week to about one month from the time of application, whereas the SDCs are prepared such that they biodegrade during a period of about up to three months from the time of application.

In still another aspect of the present invention, there is provided a method for treating a disease in a subject, said method comprising instilling into an anatomic area affected by said disease (being pre- or post-surgery) a therapeutically effective amount of a composite of a polymeric matrix embedded with at least one hydrophobic organic compound associated with at least one radioactive atom, wherein said at least one hydrophobic organic compound associated with at least one radioactive atom is substantially non-leachable from said matrix.

The invention further provides a method for the treatment of a locoregional disease after tumor resection, said method comprising instilling into an anatomic area affected by said tumor a therapeutically effective amount of a composite of a polymeric matrix embedded with at least one hydrophobic organic compound associated with at least one radioactive atom, wherein said at least one hydrophobic organic compound associated with at least one radioactive atom is substantially non-leachable from said matrix.

In one embodiment, the instillation may be in the form of a therapeutic source. The instilled composite or therapeutic source may be for example into a site of a surgically removed tumor or into or around a body organ.

As used herein, the term “treating” or any lingual variation thereof refers to the instillation, i.e. administration of the composite of the invention with the intention of preventing a disease, disorder or a certain condition associated therewith from occurring, inhibiting the disease or arresting or slowing its progression, relieving the symptoms associated with the disease, and ameliorating at least one symptom of the disease.

As used herein, the term “anatomic area” refers to any part of the body, being a tissue or an organ of the body or a cavity. The term “tissue” refers to an aggregation or collection of morphologically similar cells and associated accessory and support cells and intercellular matter, including extracellular matrix material, vascular supply, and fluids. The tissue may be any tissue of the body including blood. The term “organ” refers to any part of the body of an animal or of a human that is capable of performing some specialized physiological function. The term may include any part of such an organ or a collection of one or more of such organs. Non-limiting examples of organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow.

In one embodiment, said disease is a neoplasm, which may be benign or malignant. The neoplastic diseases generally include cancers of the prostate, lung, cervical, colorectal, pancreas, breast, head and neck, melanomas or solid tumors of soft tissues.

Non-limiting examples of neoplastic diseases are adrenocortical carcinoma, anal cancer, bladder cancer, brain tumor, brain stem glioma, cerebellar astrocytoma, cerebral astrocytoma, ependymoma, medulloblastoma, supratentorial primitive neuroectodermal and Pineal tumors, visual pathway and hypothalamic glioma, breast cancer, carcinoid tumor of the gastrointestinal, cervical cancer, colon cancer, endometrial cancer, esophageal cancer, extrahepatic bile duct cancer, Ewing's family of tumors, extracranial germ cell tumor, eye cancer, intraocular melanoma, gallbladder cancer, gastric cancer, germ cell tumor, gestational trophoblastic tumor, head and neck cancer, hypopharyngeal cancer, islet cell carcinoma, laryngeal cancer, lip and oral cavity cancer, liver cancer, lung cancer, malignant mesothelioma, melanoma, merkel cell carcinoma, metastatic squamous neck cancer, plasma cell neoplasms, mycosis, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, paranasal sinus cancer, parathyroid cancer, penile cancer, pheochromocytoma cancer, pituitary cancer, prostate cancer, rhabdomyosarcoma, rectal cancer, renal pelvis and ureter, salivary gland cancer, Sezary syndrome, small instetine cancer, soft tissue sarcoma, stomach cancer, testicular cancer, tymoma, thyroid cancer, urethral cancer, uterine cancer, vaginal cancer, vulvar cancer, and Wilm's tumor as well as metastasis of the above.

Preferably, the neoplastic disease is breast cancer, liver cancer or lung cancer, any subtype thereof as well as their metastasis.

When, for example, the breast tumor is excised surgically, the most likely site of recurrence is known to be in the region immediately surrounding the excised tumor. For such a reason, the surgery is typically followed by extension radiation therapy in this region which increases the chance of healthy tissues being damaged by the radiation. Thus, by constructing the composite of the invention into forms such as radioactive sutures, radioactive mesh, etc., a simpler and safer method of irradiation is accomplished.

Methods for treating cancers according to the present invention involve gaining access to the anatomic site where the cancer is to be treated or its evolution prevented and instilling therein the composite of the invention, a composition containing thereof or a source manufactured therefrom. The term “instilling” or any lingual variation thereof, in its broadest scope refers to any type of administration or placement of the composite of the invention in the anatomic site of a subject. Such instillation may employ any method known to a medical practitioner. Typically, access to the anatomic site may be gained by surgical or other invasive procedures, e.g. laparoscopy. Non-surgical methods for the instillation of the composite may also be employed. One such method makes use of colonoscopy for the delivery of the composite of the invention to the colon for the treatment of, e.g. colorectal cancer. In the case, for example, of skin cancers or disorders associated with the skin, the composite of the invention may be placed in direct contact with the sldn, typically without the need of employing any invasive procedures.

Generally, the composite of the invention, a composition comprising thereof or a source manufactured therefrom may be instilled into any anatomic site of the body (human or animal) by accessing the anatomic site and placing the composite of the invention therein. Depending on the method employed for accessing the anatomic site, i.e. surgical methods, non-invasive methods or non-surgical methods, further post operative methods may be employed.

The instillation allows preventing, minimizing, delaying or arresting the occurrence or recurrence of a cancer in a patient who is at risk of developing the disease. The composite, composition or source may be instilled to permanently remain in the anatomic site or degrade over time, resorbed by the tissues and metabolized. Repeated instillations of the composite, composition or source may be undertaken based on the specific formulations.

Combination therapies for advanced cancer patients may also be envisioned and thus fall within the scope of the present invention. Some combination therapies may involve the instillation of a composite of the invention into the anatomic site simultaneously with the administration of another treatment such as systemic chemotherapy, locoreginal radiation therapy, cryotherapy, resection of tumors, and others.

The composite of the invention may also be used in non-neoplastic diseases and conditions such as restenosis. Restenosis is the narrowing of a blood vessel (usually a coronary artery) following the removal or reduction of a previous narrowing (angioplasty). Due to cell proliferation and/or plaque formation occurring in more than 40% of all post-angioplasty procedures, physicians are forced to perform complex and life threatening procedures such as coronary artery heart bypass surgery. The composite of the invention may be utilized in such cases as well as, for example, a stent-like radiation source for the delivery of a dose of radiation to the artery walls. This direct and local radiation assists in reducing the chance of restenosis and the likelihood for more complex and dangerous post-angioplasty procedures.

### BRIEF DESCRIPTION OF THE DRAWINGS

In order to understand the invention and to see how it may be carried out in practice, preferred embodiments will now be described, by way of non-limiting examples only, with reference to the accompanying drawings, in which:

FIG. 1 demonstrates the kinetics of eosin adsorption onto the chitosan (Ct) gel G12.5 (A) and the effect of increasing glutaraldehyde (GA) concentrations (as expressed by the GA: Ct ratios) on the crosslinking density as expressed by the eosin adsorption (B). Shown are the mean values of four measurements±SD.

FIG. 2 demonstrates the enthalpy (A) and Tg (B) of Ct gels with increasing ratios of GA: Ct as computed from DSC curves.

FIG. 3 demonstrates the elasticity of the various Ct gels with increasing ratios of GA: Ct. Shown are the mean values of 6 different measurements±SD.

FIG. 4 demonstrates the effect of ionic strength (mM of sodium chloride-filled circles), osmolarity (mM of glucose—empty circles) (A) and pH (B) of the external medium on the swelling of G10. Shown are the mean values of 4 different measurements±SD.

FIG. 5 demonstrates the in vivo degradation of SDC (empty circles) and FDC (filled circles) after SC (A) and IP (B) implantation in the rat. Shown are the mean values of 4 different experiments A SD.

FIG. 6 demonstrates the in vivo release of SB from SDC (empty circles) and FDC (filled circles) after SC (A) and IP (B) implantation in the rat. Shown are the mean values of 4 different experiments z SD.

FIG. 7 demonstrates representative gamma camera following the implantation of SDC loaded with ¹³¹I-NC at 0, 4, 13 and 30 days after implantation (A). Percent of initial activity at the site of implantation (filled circles) and in the axillar lymph nodes (empty circles) after implantation of SDC loaded with ¹³¹I-NC in the left pectoral region of the rat. Shown are the mean values of at least 3 different experiments±SD (B).

FIG. 8 demonstrates representative histological sections demonstrating minimal tissue response in the tissues surrounding SDC implant, original magnification ×100 (A), FDC implant, original magnification ×100 (B), and chronic foreign body reaction in the tissues surrounding surgical sutures, original magnification ×200 (C).

FIG. 9 demonstrates representative histological sections of tissues surrounding FDC implants 1 (A), 3 (B), 7 (C) and 14 (D) days after subcutaneous implantation and 14 days after intraperitoneal implantation (E). PMN-Polymorphonuclears, AF—Activated fibroblasts, L—Lymphocytes, BV—Newly formed blood vessels, M—Macrophages, FC—Fibrous capsule. Magnification: A and B×200, C, D and E×100.

FIG. 10 demonstrates representative histological sections of tissues surrounding SDC implants 3 (A), 7 (B), 14 (C) and 28 (D) days after subcutaneous implantation and 28 days after intraperitoneal implantation (E). FC—Fibrous capsule. Magnification: A, B and E×200, C, and D×100.

FIG. 11 demonstrates comparison of tissue response to: (A) polyglycolic-polylactic absorbable suture after 28 days of subcutaneous implantation, (B) SDC after 28 days of subcutaneous implantation and (C) FDC after 14 days of subcutaneous implantation. S—Suture fibers, PR—Particulate response. Magnification: A×200, B and C×100.

FIG. 12 demonstrates partial degradation of the FDC gel after intraperitoneal implantation at day 7 (A); its complete degradation after subcutaneous implantation at day 14 (B); and FDC debris in macrophages in the hillum of a lymph node (C). ND-Non degraded gel, PD—Partially degraded gel, MD—Mostly degraded gel, M-Macrophage loaded with FDG debris. Magnification: A×200, B and C×100.

FIG. 13 demonstrates adsorption of hematoxylin (open circles) and eosin (closed circles) onto the FDC implant after in vitro oxidation with increasing concentrations of KMnO₄. Shown are the mean of 5 measurements±S.D.

FIG. 14 demonstrates representative histological sections showing fragments of the of ¹³¹I-NC loaded SDC implants (A) and the damage caused to the surrounding tissues, 28 days after subcutaneous implantation, as detected by massive infiltration of inflammatory cells and absence of tissue integrity (B). PR—Particulate response, MF—Muscular fibers. Magnification: A×50, B×100.

FIG. 15 demonstrates tumor progression as expressed by tumor weight in the non-treated control group, open squares; implantation of empty hydrogels group, open-circles; and implantation of ¹³¹I-NC loaded hydrogels group, filled circles. Shown are the mean values of 3 different experiments±SEM.

FIG. 16 shows representative images of different tumors removed two weeks post implantation (A-C).

FIG. 17 shows the time to death in tumor progression model. Kaplan-Meier survival curves for non-treated control group, discontinuous line; implantation of empty hydrogels group, continuous line; and implantation of ¹³¹I-NC loaded hydrogen group, bold continuous line.

FIG. 18 shows the time to death in micro-residual disease model. Kaplan-Meier survival curves for non-treated control group, discontinuous line; implantation of empty hydrogels group, continuous line; and implantation of ¹³¹I-NC loaded hydrogels group, bold continuous line.

FIG. 19 shows representative images of mice (A, B, C) at week 10 weeks after beginning of the treatment in micro-residual disease model: non-treated control group, A; implantation of empty hydrogels group, B; and implantation of ¹³¹I-NC loaded hydrogels group, C.

FIG. 20 shows the histopathological analysis of the specimens taken from tumor bed and distant organs, exemplifying distant organs such as lung and liver.

FIG. 21 demonstrates the activity of ¹³¹I-NC loaded hydrogels after subcutaneous implantation in the back of mice. Calculated physical decay (discontinuous line) and actual bio-physical decay (continuous line) of ¹³¹I-NC loaded hydrogels after subcutaneous implantation in the back of mice. Shown are the mean values of 4 different experiments±SEM.

### DETAILED DESCRIPTION OF THE INVENTION

A person of skill in the art would recognize that the examples provided herein are presented as non-limiting embodiments of the present invention. Thus, for example, a person skilled in the art would be of the knowledge to replace one polysaccharide under another employing the necessary modifications.

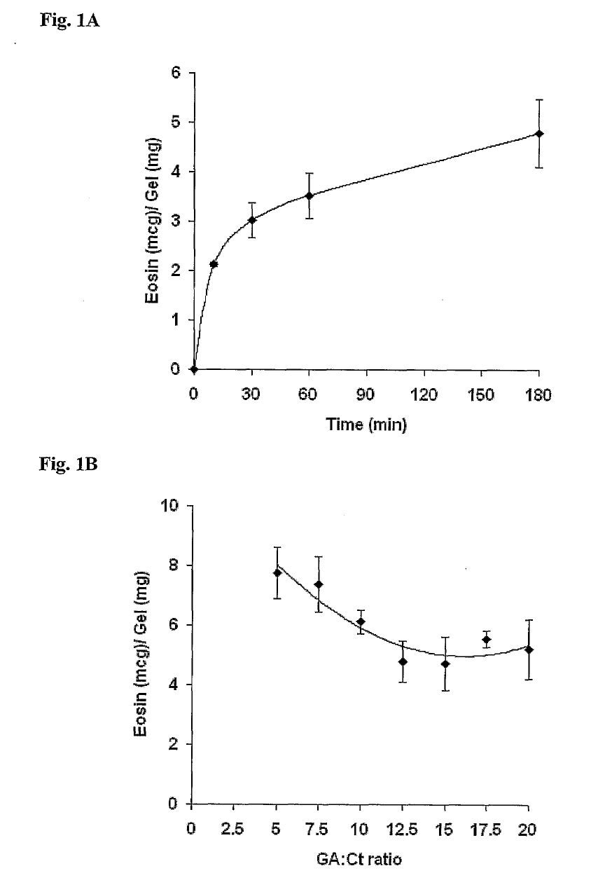

Increasing amounts of glutaraldehyde (GA) were used to prepare a series of chitosan (Ct) hydrogels with different crosslinking densities that were characterized by eosin adsorption. Typically, the adsorption process lasted about three hours (FIG. 1A) and the amount of eosin adsorbed into the gels was inversely proportional to the relative amount of GA used for crosslinking; the higher the ratio of GA to Ct, the lower the amount of eosin adsorbed. Minimal adsorption was observed at GA:Ct ratios of 10-12.5:1 (FIG. 1B), indicating that the reaction reached its end-point with this quantity of GA.

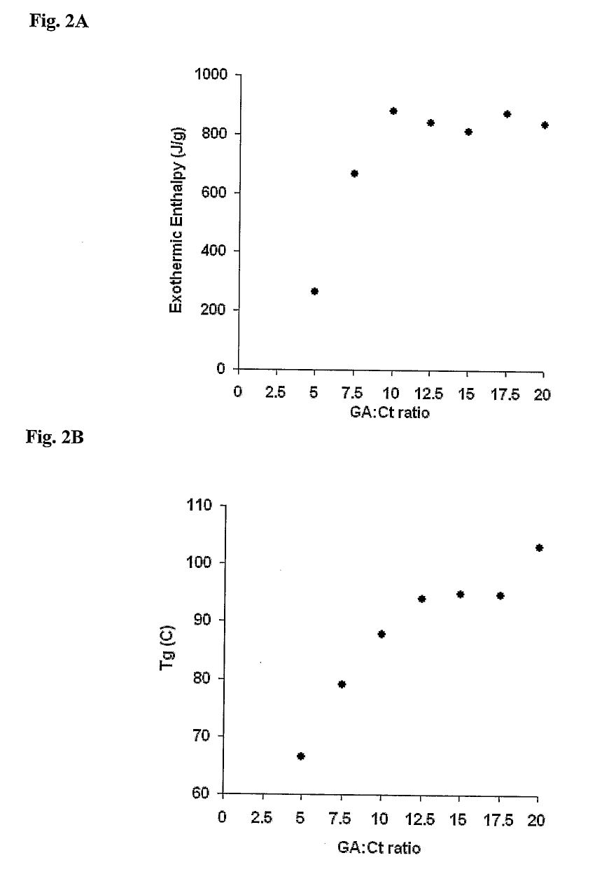

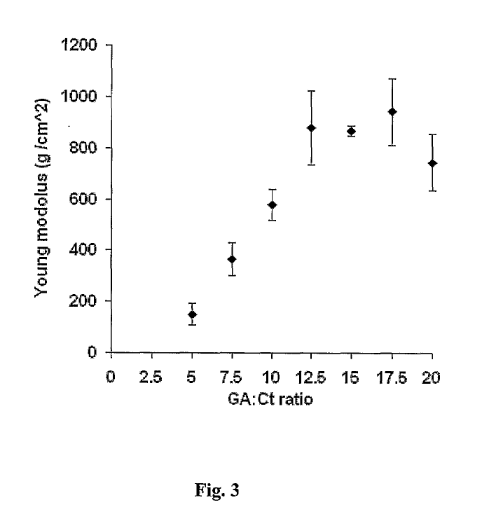

Differential Scanning Calorimetry (DSC) analysis provided information about the thermal changes of the hydrogels, from which the exothermic enthalpy and T_{g} was computed. FIG. 2A shows a direct correlation between the ratio of GA in the reaction mixture and the exothermic enthalpy, and that the ratio of 10:1 (herein referred to as product G10) was the upper limit for the crosslinking reaction. Similar correlation was demonstrated by T_{g} measurements (FIG. 2B). The Young Modulus was calculated from the elasticity studies (FIG. 3) and demonstrated that the GA ratio of 12.5:1 was the upper limit of the crosslinking reaction.

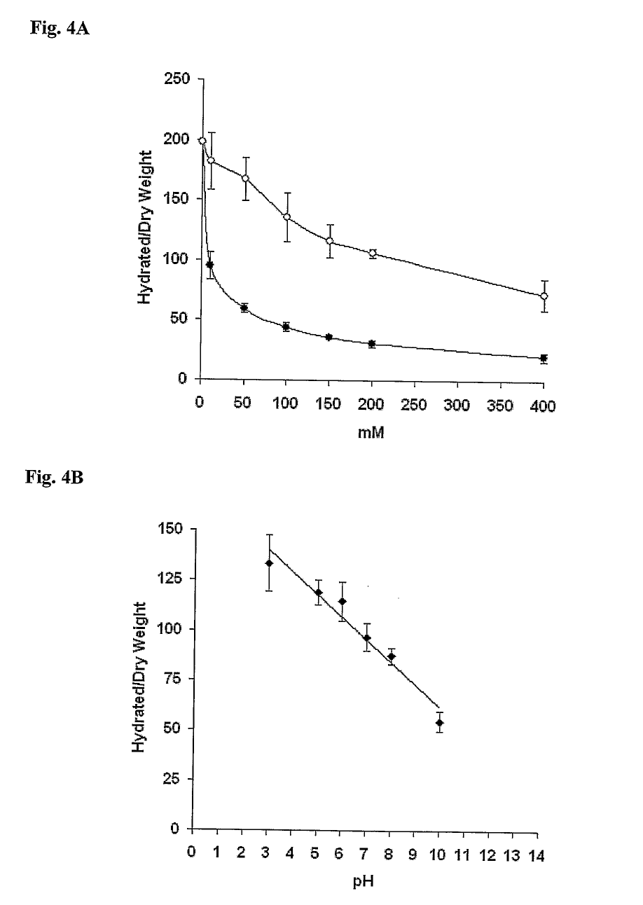

The swelling of G10 gel in increasing ionic strength, osmolarity and pH is depicted in FIG. 4. Inverse proportionality was observed between the swelling of the gel and the ionic strength, osmolarity (FIG. 4A) and pH (FIG. 4B), with the most profound effect achieved by changes in ionic strength.

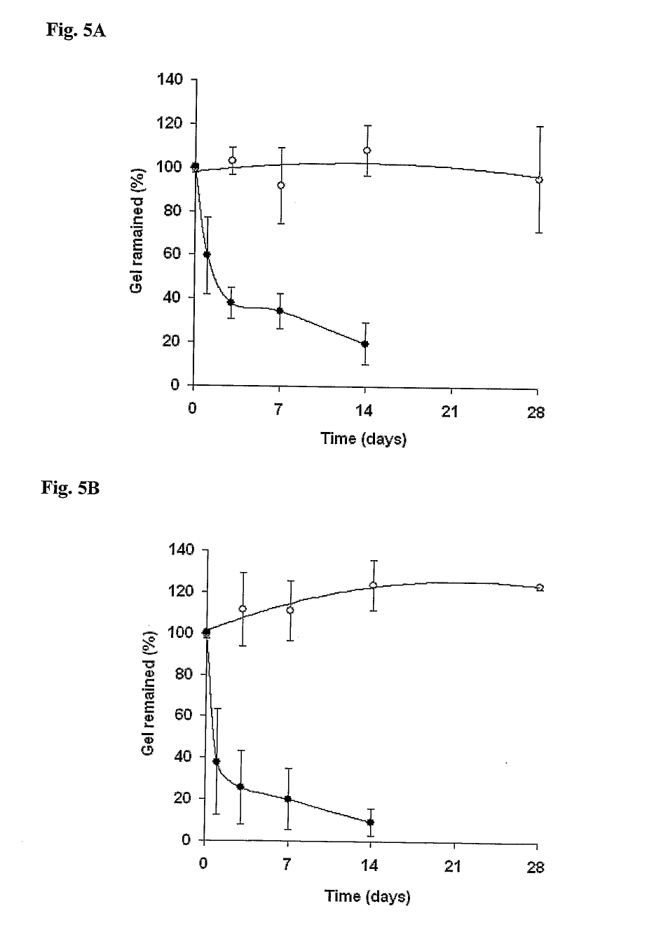

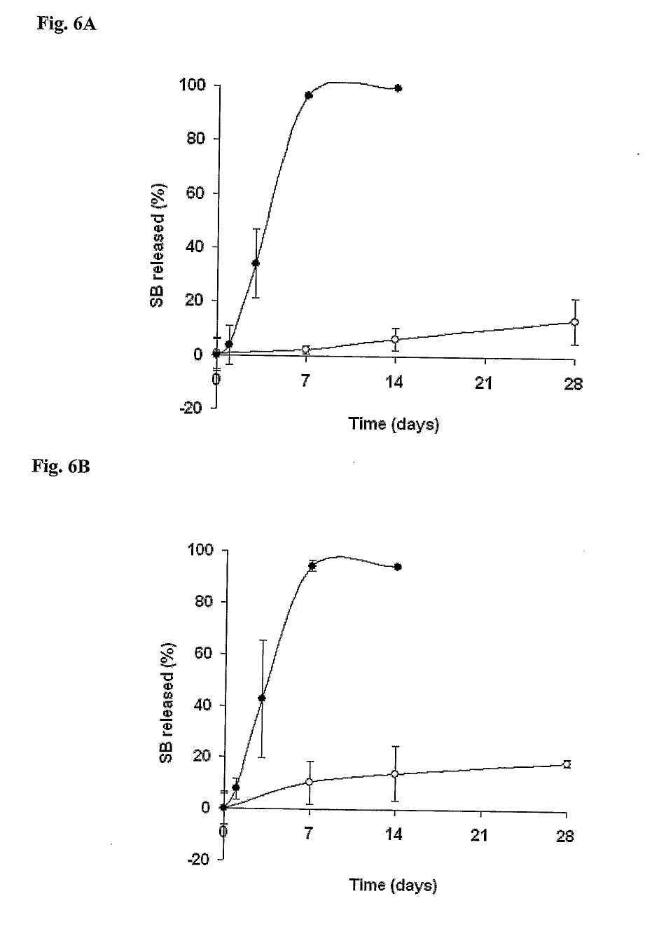

G10 was further used to produce two types of implants, slow degrading composite (SDC) and fast degrading composite (FDC) of G10, differing from each other by their in vivo degradation rates. The former was obtained by dialysis against PBS and the latter was dialyzed against water, which resulted in different swelling properties. The degradation properties of the two gels were tested following SC and IP implantation in rats. No weight loss of the SDC could be detected over 28 days for both SC and IP implantations. In contrast, only 19.8±9.5 and 9.2±6.5% of the FDC was lest after 14 days of SC and IP implantations, respectively (FIG. 5). To verify the degradation results and to investigate the gels' ability to serve as platforms for hydrophobic probes, both types of implants were loaded with Suddan Black (SB). The study of the SB release kinetics in vivo revealed that only 13.6±8.3 and 18.7±1.4% SB were released from the SDC after 28 days of SC and IP implantations, respectively. However, almost complete SB release occurred during the first week of and IP implantation of FDC, indicating that its degradation was the cause for the accelerated release of the dye (FIG. 6).

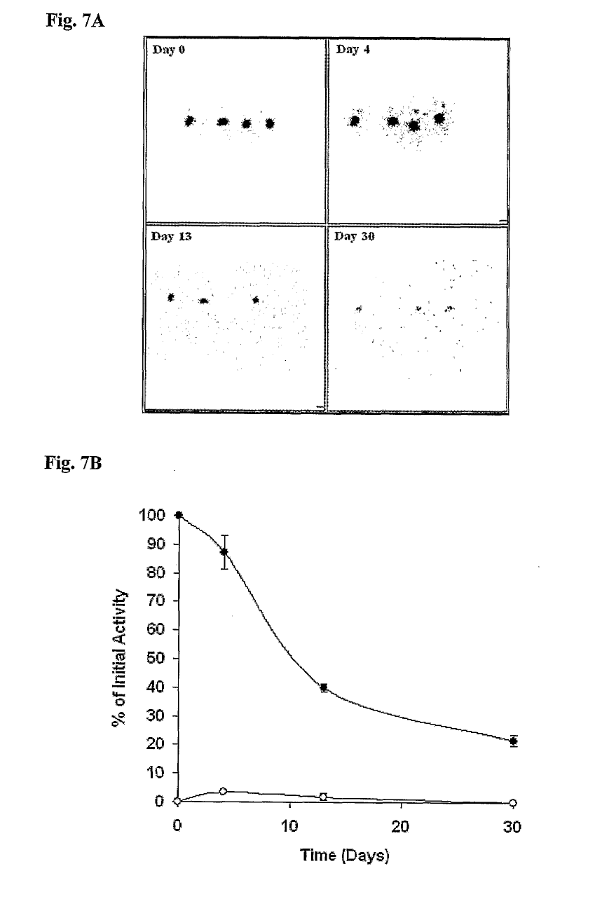

FIG. 7A shows representative examples of scintigraphy images from rats after implantation of SDC containing ¹³¹I-NC at different time points, FIG. 7B shows the distribution of ¹³¹I-NC after implantation at these time points. It was found that 80% of the ¹³¹I-NC was released from the implant 30 days after implantation and that 4% was found in the axillar lymph nodes at days 4 and 13.

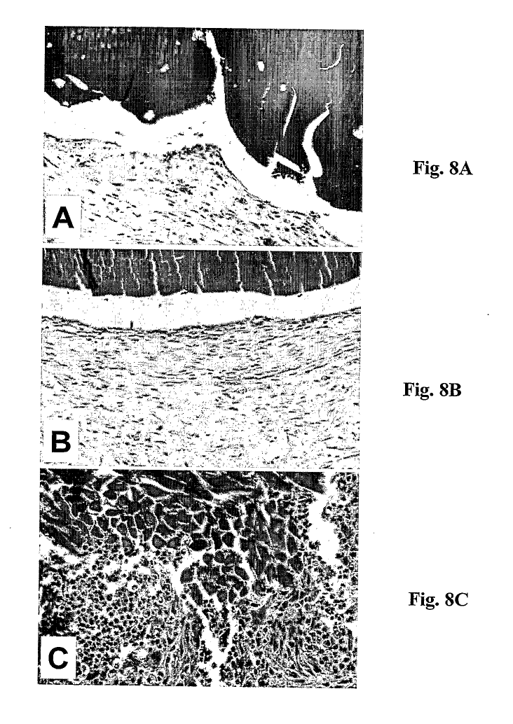

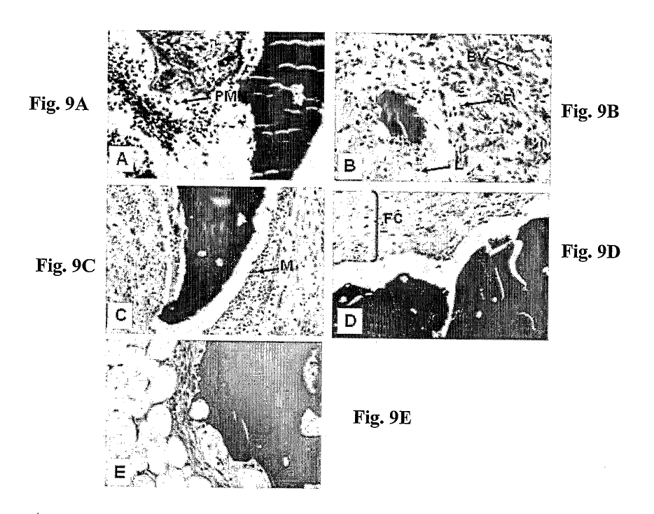

Histological observations shown in FIG. 8 clearly demonstrate that 14 and 28 days after implantation of the FDC and SDC, respectively, the implants were encapsulated within a fibrotic capsule, with minimal inflammatory cellularity and occasional capillaries transecting the fibrous tissue. The average thickness of the peri-implant capsules was 80-100 micrometer in both cases (FIG. 8 panels A and B). At the time of sampling, partial degradation of both FDC and SDC was observed with a greater extent of decomposition exhibited by the FDC implant (data not shown). Interestingly, the biodegradable surgical suture developed a typical chronic foreign body reaction (inflammation) with high numbers of polymorphonuclears, lymphocytes, macrophages and foreign body giant cells (Panel C of FIG. 8). This is in contrast to the histological findings of the specimens taken from the implant regions of the FDC and SDC implants (FIGS. 9 to 14), as will be shown in the Examples.

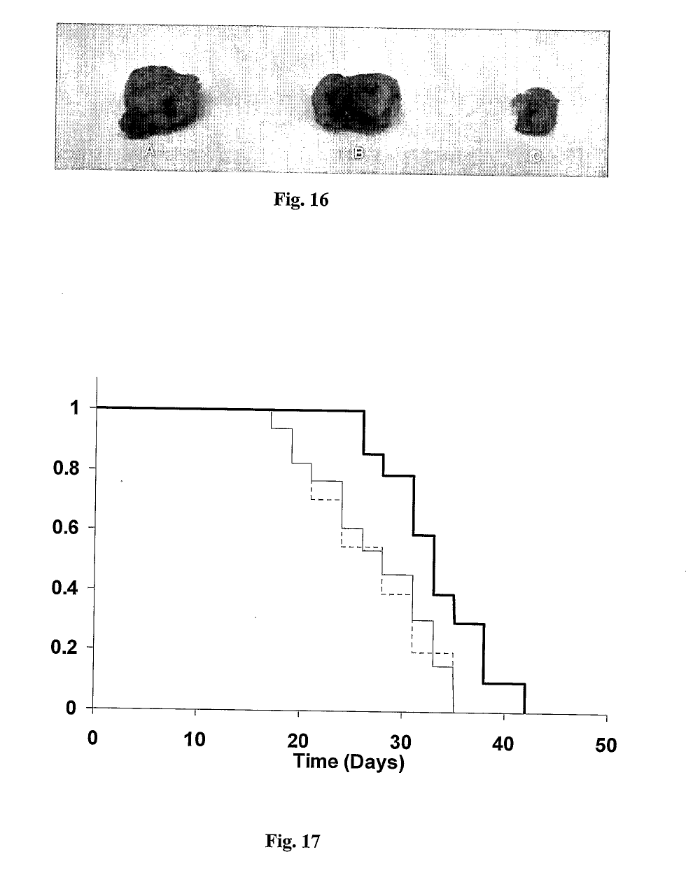

The implantation of the ¹³¹I-NC loaded implants was shown to delay the progression of solid tumors, as shown in FIG. 15. There was a significant difference in tumor growth rate between the group treated with ¹³¹I-NC loaded implant and the non-treated and empty implant treated groups during the first two weeks after hydrogel implantation. The most significant difference was achieved by the end of the second week, when the tumor volume in the group treated with ¹³¹I-NC loaded implant was approximately 72% of the volume in the other groups (FIG. 16A-C). Moreover, there was a significant macroscopic difference in the number of metastatic nodes in the lung. These findings are especially relevant when compared with the ineffectiveness of external beam radiation, and the only 50% inhibition of growth by a combination of radiation and enhancing factors for ionizing radiation in the 4T1 xenograft model.

Kaplan-Meier analysis of mice survival of the different groups showed increased survival of the treated group by 120% compared to control groups (42 and 35 days respectively) (FIG. 17). These results suggest that treatment with hydrogel implants loaded with ¹³¹I-NC is comparable to treatment with external beam radiation, which increased survival of mice in the 4T1 xenograft model by 112% as compared to control groups (45 and 40 days respectively).

The long-term survival rate among women who undergo breast-conserving surgery is the same as that among women who undergo radical mastectomy. Moreover, the cumulative incidence of locoregional tumor recurrent in breast was significantly decreased in women who underwent lumpectomy and breast irradiation, as compared with women who underwent lumpectomy without irradiation.

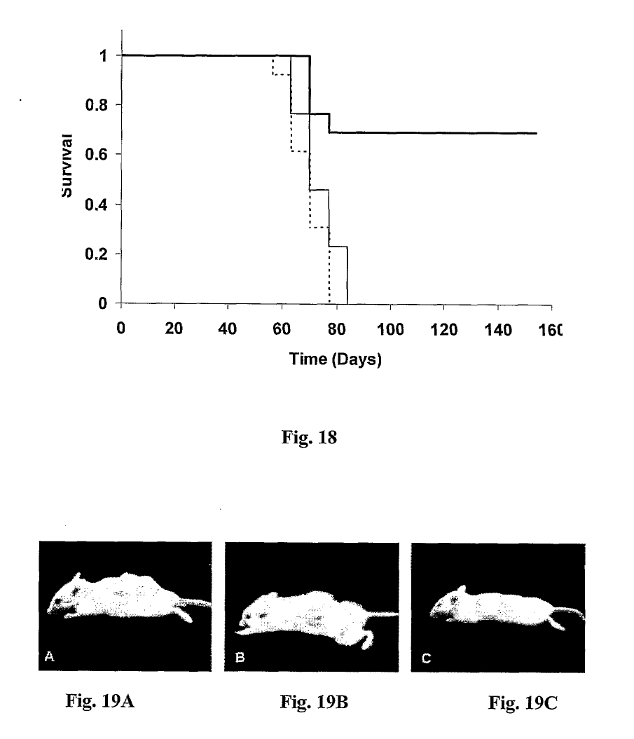

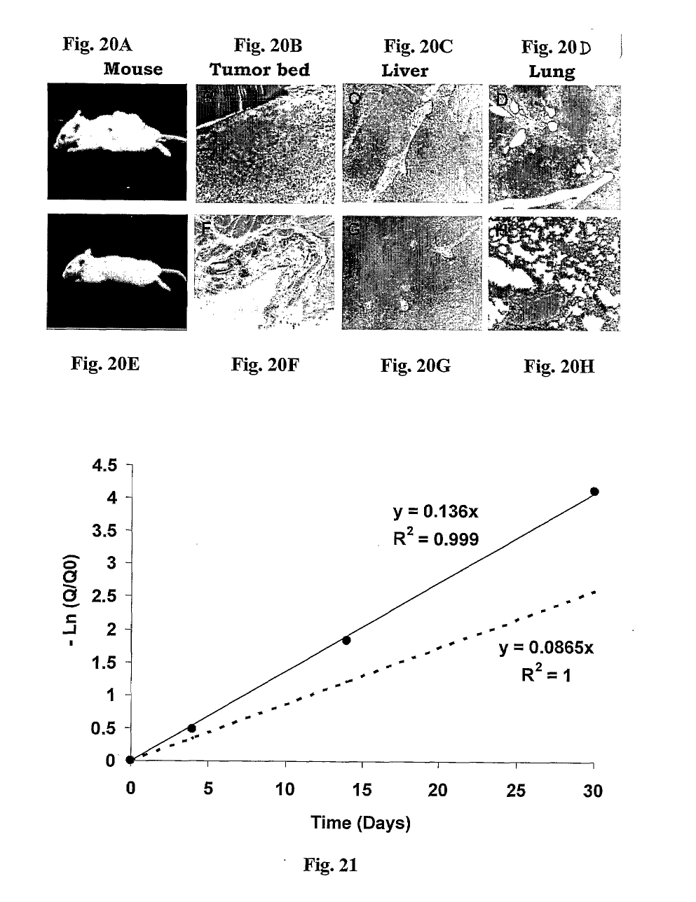

On this background, the effect of the composites of the invention loaded with ¹³¹I-NC in the prevention of locoregional recurrence in xenograft breast cancer model was assessed. During surgery to implant the composites of the invention, 4T1 cells (10% of the amount needed for primary tumor induction) were spread subcutaneously in the surgical cavity, to mimic cancer cell spillage in the tumor bed causing the locoregional recurrence. Kaplan-Meier analysis showed that the survival of the group treated with hydrogels loaded with ¹³¹I-NC was 69.2%, as compared to death of all mice in the group treated with empty gels and in the untreated group (FIG. 18). Macroscopically, there were no signs of tumor in the group treated with hydrogels loaded with ¹³¹I-NC after 77 days, as compared to large tumors developed in the group treated with empty gels and in the untreated group (FIG. 19). Histopathological analysis of the specimens taken from tumor bed and distant organs from these groups did not show signs of tumor or metastasis progression, neither in the tumor bed nor in the distant organs (FIG. 20A-E). In contrast, tumors and metastasis in lung, heart, liver and spleen developed in the untreated group (FIG. 20F-J) and the empty hydrogel treated group (FIG. 20F-J)

Without wishing to be bound by theory, the elimination of the radioactivity from the site of implantation in vivo is a result of two parallel pathways: the radioactivity decay and the biological elimination of the radioactive material. It was earlier shown that the elimination of ¹³¹I-NC depended on the degradation of the hydrogel, due to the hydrophobic nature of the ¹³¹I-NC and the hydrophilic nature of the hydrogel. The total radioactivity elimination constant was calculated as the slope of linear regression of the natural logarithm of the remaining fraction of radioactivity with time (FIG. 21, continuous line). The radioactivity elimination constant consists of the radioactive decay constant and the biological elimination (hydrogel degradation) constant. The radioactivity decay constant can be calculated from the radioactivity decay half-life or alternatively to be obtained from the slope of the linear regression of the natural logarithm of the theoretical fraction of radioactivity remaining after radioactive decay of the isotope with time (FIG. 21, discontinuous line). The hydrogel degradation constant was calculated by subtraction of the radioactive decay constant from the total elimination constant. The hydrogel degradation half life was calculated from the hydrogel degradation constant to be 14.0 days. The biological degradation appears fit as a first order kinetics, due to its compatibility to the equations of the first order kinetics Eq. 5, 6 and 7 (see Examples), showed in the fit of the experimental data with the linear regression expressed by the regression coefficient (R²=0.999). This method for estimating hydrogel degradation kinetics is more accurate than the traditional gravimetric methods, since in gravimetric methods, residual hydrogels are retrieved from animals, dried and weighted to calculate the remaining fraction of the gel. In the traditional gravimetric methods there are some major constraints such as incomplete retrieving of the gels, tissue penetration in the hydrogels, variations in the drying of the gels and inaccurate weighting of the hydrogels especially for low weights (few milligrams) of the dry hydrogels. The current radioactivity decay method overcomes these constraints by delivery of actual time processes in the animal with no need for hydrogel retrieval, drying or weighing. Moreover, this method takes into consideration structural changes that may cause drug release without weight change of the implant.

In summary, implantation of composites of the invention such as hydrogels loaded with ¹³¹I-NC in the vicinity of tumor, reduced tumor progression rate by two weeks compared with the control groups. Implantation of the hydrogels loaded ¹³¹I-NC in the tumor bed in a residual disease model prevented tumor recurrence and increased survival by 69% as compared with a complete mortality of the control groups. Histopathological analysis of the tumor bed and distant organs after implantation of the hydrogels showed that the implants of the invention were safe and biocompatible.

A—Biocompatibility Evaluation and Mode of Degradation of Crosslinked Chitosan Hydrogels after Subcutaneous and Intraperitoneal Implantation in Rats

### Example 1

### Preparation of the Chitosan (Ct) Gels

One hundred milligrams of Ct was dissolved in 10 ml of 1M acetic acid (Frutarom, Israel), and heated to 100° C. Glutaraldehyde (GA) solution (25% w/v in water) was then added while stirring. A gel was formed immediately and the stirring was stopped. Assuming complete reaction between one molecule of GA and two glucosamine repeating units, the following Ct:GA molar ratios were examined in different studies: 1:5, 1:7.5, 1:10, 1:12.5, 1:15, 1:17.5 and 1:20. These ratios are hereby denoted as G5, G7.5, G10, G12.5, G15, G17.5 and G20 respectively. Excess of GA was removed by dialysis until no traces of GA could be detected at 235 nm (polymeric GA) and 280 nm (monomeric GA) (Uvikon 930, Kontron Instruments, Switzerland) in the rinsing medium.

### Example 2

### Crosslinking Density Characterization

The crosslinking density of the gels was quantified by adsorption measurements of the negatively charged dye eosin from a hydroalcoholic solution. In different studies about 0.2 g of each gel was incubated in 2 ml of 0.05 mg of eosin in ethanol:water 1:1 solution for 10, 30, 60 and 180 minutes at room temperature. The gels were then removed and the eosin concentration in the incubation medium was measured spectrophotometrically (520 nm), using a six-point calibration curve. The gels were then rinsed with water, dried in acetone (48 hours) and weighed. The amount of eosin adsorbed, which was calculated from the initial and final concentrations in the bathing solution, was normalized to the dry weight of each gel.

The gels' crosslinking density was also characterized by differential scanning calorimetry (DSC) analysis. The change in heat capacity of the pre-dried gels was measured in a temperature range of 25-175° C., at a rate of 10° C./min, under N₂ flow of 1 ml/min (Mettler Instruments, Switzerland, TA 4000, equipped with TC II TA processor). DSC curves were plotted and the glass transition temperature (Tg) and enthalpy (ΔH) were computed by the apparatus program.

Typically, the adsorption process lasted about three hours (FIG. 1A) and the amount of eosin adsorbed into the gels was inversely proportional to the relative amount of GA used for crosslinking; the higher the ratio of GA:Ct, the lower the amount of eosin adsorbed. Minimal adsorption was observed at GA:Ct ratios of 10-12.5:1 (FIG. 1B), indicating that the reaction reached its end-point with this quantity of GA.

DSC analysis provided information about the thermal changes of the hydrogels, from which the exothermic enthalpy and Tg was computed. FIG. 2A shows a direct correlation between the ratio of GA in the reaction mixture and the exothermic enthalpy, and that the ratio of 10:1 (product G10) was the upper limit for the crosslinking reaction. Similar correlation was demonstrated by Tg measurements (FIG. 2B).

### Example 3

### Mechanical Properties Characterization

Cubic (S-4 mm) specimens from each gel were cut by a scalpel and tested in a texture analyzer (TAXT Plus, Texture Technologies, USA), at a rate of 0.05 mm×sec⁻¹ (compression). Young's modulus of elasticity (E) was calculated using to the following equation:

E=(F/A)/(ΔL/L₀) Eq. 1

where F is the tensile force (in gF), A is the cross section area of the specimen (cm²), ΔL is the specimen's strained length (mm) and L₀ is the initial length of the gel's specimen (mm).

The Young Modulus was calculated from the elasticity studies (FIG. 3) and demonstrated that the GA ratio of 12.5:1 was the upper limit of the crosslinking reaction.

### Example 4

### In Vitro Swelling Studies

Since the gels are designed to be implanted in the hydrated form, the effect of ionic strength, osmolarity and pH on their swelling properties were studied in vitro. In separate studies specimens of G10 were incubated in increasing concentrations (0, 10, 50, 100, 150, 200 or 400 mM) of NaCl (increasing ionic strength) or glucose (increasing osmolarity). Similarly, the G10 specimens were incubated in 10mM phosphate buffer at different pH values (3, 5, 6, 7, 8 and 10). The incubation lasted 12 hours, after which time the specimens were rinsed with water, weighed (W_{S}), dried, weighed again (W_{D}) and the W_{S}/W_{D} ratio was calculated.

The swelling of the G10 gel in increasing ionic strength, osmolarity and pH (expressed as the ratio of its weight in a swollen state to its dry weight) is depicted in FIG. 4. Inverse proportionality was observed between the swelling of the gel and the ionic strength, osmolarity (FIG. 4A), and pH (FIG. 4B), with the most profound effect achieved by changes in ionic strength.

### Example 5

### In Vivo Degradation Studies

Two formulations of G10 were prepared and each was subjected to a different mode of dialysis after crosslinking. The slow degrading G10 (SDC) was prepared by dialysis against PBS (1 mM, pH=7.4), while the fast degrading G10 (FDC) was prepared by dialysis against water.

In separate studies the SDC and FDC were implanted both intraperitonealy (IP) and subcutaneously (SC) in the rat. The former was conducted by laparotomy through a midline incision in the anesthetized rat, and placement of the gel specimen between the intestine and the peritoneum, approximately 1 cm left to the incision. The latter was conducted by retracting both muscles and skin to form a cavity into which the gel specimen was inserted, approximately 1 cm left to the incision. The abdominal cavity and skin were then closed using a 3-0 vicril running suture (Johnson & Johnson Medical). Following surgery the rats were supervised until complete recovery and then normal diet was resumed. At 0, 1, 3, 7 and 14 days, for the FDC, and 0, 3, 7, 14 and 28 days for SDC, four rats from each group were sacrificed. The gels were retrieved, rinsed in water, dried and weighed (W_{Rem}).

The extent of the in vivo degradation (as % of initial amount) was assessed from the ratio of the gels' dry weight before and after implantation (the gels were implanted in the hydrated form and lost water during the course of the rat studies). The weight ratios hydrated/dry of the gels prior to implantation were measured and found to be 52.0±0.9 and 198.1±1.9 for the SDC and FDC, respectively. W₀ was calculated from the above ratios. The extent of gel degradation, in percent of initial amount (% Remained), was calculated using the following equation:

% Remained=(W_{Rem}/W₀)×100 Eq. 2

where, W_{Rem} is the dry weight of the gel debris retrieved at the end of each implantation study and W₀ is the initial dry weight of the respective gel.

G10 was further used to produce two types of implants, SDC and FDC, differing from each other by their in vivo degradation rates. The former was obtained by dialysis against PBS and the latter was dialyzed against water, which resulted in different swelling properties (wet/dry weight ratios of 198.1±1.9 compared with 52.0±0.9, respectively). The degradation properties of the two gels were tested following SC and IP implantation in the rat. No weight loss of the SDC could be detected over 28 days for both SC and IP implantation. In contrast, only 19.8±9.5 and 9.2% 6.5% of the FDC was left after 14 days of SC and IP implantation, respectively (FIG. 5).

### Example 6

### Sudan Black Loading and In Vivo Release Kinetics

The hydrophobic dye Sudan black (SB) was loaded into the SDC and FDC gels to allow further insight into the degradation kinetics of the two types of implants in vivo. SB was dispersed in the acidic Ct solution to obtain a final SB:CT ratio of 1:100. After crosslinking with GA to obtain an SB loaded G10 hydrogel, SB loaded SDC and FDC gels were prepared by dialysis, as described above. After weighing, the two products were implanted SC and IP in the anesthetized rats. The rats recovered and were maintained with free access to normal rat chow and water. At 0, 1, 3, 7 and 14 days, for the FDC implanted group, and 0, 3, 7, 14 and 28 days for SDC implanted group, four rats were sacrificed at each time point. The gels or gel debris were located, separated from the tissues, rinsed and soaked in acetone for 48 h in a sealed beaker. The concentration of the extracted SB (SB_{Rem}) was measured spectrophotometrically at 600 nm and the fraction of SB released during implantation was calculated. The initial amount of SB in the gels (SB₀) was determined by acetone extraction at time 0. The amount of SB remaining in each gel specimen removed from the rats at each time point was measured similarly and normalized to the gel weight. SB₀ was calculated from the weight of the implanted gel and the calculated SB/Gel ratio, while the fraction of SB released from the gel was calculated as follows:

% SB Released=(SB₀−SB_{Rem})/SB₀×100

To verify the degradation results and to investigate the gels' ability to serve as platforms for hydrophobic probes, both types of implants were loaded with SB. The study of the SB release kinetics in vivo revealed that only 13.6±8.3 and 18.7±1.4% SB were released from the SDC after 28 days of SC and IP implantation respectively. However, almost complete SB release occurred during the first week of SC and IP implantation of FDC, indicating that its degradation was the cause for the accelerated release of the dye (FIG. 6).

### Example 7

### ¹³¹I-Norcholesterol (¹³¹I-NC) Loading and Implantation in Vivo in Rats

0.2 ml of ¹³¹I-NC (0.2 mCi) (CIS Bio International, France) was dispersed in 10 ml of Ct solution (1% in acetic acid 1M), heated to 100° C. and 1.2 ml glutaraldehyde solution (25% w/w) was added to form a gel. Gels were dialyzed against PBS pH 7.4 (1 mM) for 24 hours to obtain the SDC gels, and 0.5 g specimens of the gels were implanted in the left pectoral region of the rat. Scintigraphy was performed at 0, 3, 13 and 30 days after implantation. Each rat was imaged for 15 min under anesthesia, using a helix dual-head camera (Elscint, Haifa, Israel) and a high-energy, high-resolution collimator. Data was analyzed on a Xeleris program (GE Healthcare), and regions of interest were drawn on each focus. The total number of counts in each region was calculated and percent of activity in each regions of interest was calculated as follows:

% Activity=(Aₜ/2^{-t/8.02)}/A₀×100 Eq. 4

where t=time (days), Aₜ=counts at t, A₀=counts at t₀, and 8.02 days is the half-life of ¹³¹I.

FIG. 7A shows representative examples of scintigraphy images from rats after the implantation of SDC containing ¹³¹I-NC at different time points (0, 4, 13 and 30 days). FIG. 7B shows the distribution of ¹³¹I-NC after implantation at these time points. It was found that 80% of ¹³¹I-NC was released from the implant 30 days after implantation, and that about 4% was found in the axillar lymph nodes at days 4 and 13.

### Example 8

### Safety Studies

The effect of the two types of implants, FDC and SDC on the surrounding tissues was examined histologically. Tissue specimens taken at the site of implantation were collected at 14 days for FDC and 28 days for SDC. All specimens included debris of the implant itself.

The specimens were fixed in 4% buffered formaldehyde, dehydrated, embedded in paraffin blocks and four-micron slices were sectioned and stained with hematoxylin and eosin. The sections were examined microscopically for a possible inflammatory response and evaluation of the thickness of the peri-implant fibrotic capsule. Tissues specimens containing debris of Vicryl™ biodegradable sutures served as controls for foreign body tissue interactions and were collected after 28 days.

Histological observations shown in FIG. 8 clearly demonstrate that 14 and 28 days after implantation of the FDC and SDC, respectively, the implants were encapsulated within a fibrotic capsule, with minimal inflammatory cellularity and occasional capillaries transecting the fibrous tissue. The average thickness of the peri-implant capsules was 80-100 micron in both cases (FIGS. 8 A and B). At the time of sampling, partial degradation of both FDC and SDC implants was observed with a greater extent of decomposition exhibited by the FDC implant (data not shown). The biodegradable surgical suture examined developed a typical chronic foreign body reaction (inflammation) with high numbers of polymorphonuclears, lymphocytes, macrophages and foreign body giant cells (FIG. 8C).

### Example 9

### Adjacent Tissue Response Analysis

In separate studies SDC and FDC (1 g/Kg body weight) were implanted each intraperitonealy (IP) and subcutaneously (SC) in the rat. IP implantation was conducted by laparotomy and placement of the gel approximately 1 cm left to the midline, in the peritoneal cavity. SC implantation was conducted by retracting the muscles and skin, and mounting the gel approximately 1 cm left to the midline, between the muscles and the skin. After the gels' implantation the abdominal cavity and skin were sutured and the rats were allowed to recover. At 0, 1, 3, 7 and 14 days, for the FDG implanted group, and 0, 3, 7, 14 and 28 days for SDG implanted group 4 rats were sacrificed and tissue specimens from the tissues surrounding the implants were rinsed with PBS, fixated with 4% formaldehyde in PBS, dehydrated, embedded in paraffin blocks, sectioned (4 μm) and stained with hematoxylin-eosin for histological examination of tissue reaction.

A minimum of three serial sections of each block was examined microscopically in search of cellular inflammatory response and for measurement of peri-implant fibrotic capsule thickness. The extent of inflammatory response was quantified by assessing the presence of inflammatory cells (polymorphonuclears, lymphocytes, macrophages and foreign body giant cells), fibrin, exudate, necrosis and vascularization. The presence of the above inflammatory markers was scored from (−)=absence, to (+++)=profound presence (Table 1). The peri-implant fibrotic capsule thickness was defined as the distance between the border of the fibrotic tissue, adjacent to the implant, and the muscle or fat tissue adjacent to the fibrotic capsule at the other end. The draining regional lymph nodes were microscopically examined in a search for non-typical reactive response (lymphocytes or macrophages infiltration).

After implanting the two types of gels in two different locations in the rat, inflammation was observed in the tissues surrounding the implants. The variable degrees of inflammatory component infiltration and fibrous capsule formation are summarized in Table 1, which also shows that neither hemorrhage nor necrosis occurred around the implants.

TABLE 1
Inflammation severity scoring as assessed by various
markers at 1, 3, 7 and 14 days (for FDC) and 3, 7,
14 and 28 days (for SDC) after subcutaneous (SC) and
intraperitoneal (IP) implantation in the rat.

| | | Fast Degrading Gel | | | | Slow Degrading Gel | | | |
|---|---|---|---|---|---|---|---|---|---|
| Time (days) | | 1 | 3 | 7 | 14 | 3 | 7 | 14 | 28 |
| Exudate | SC | + | − | − | − | ± | − | − | − |
| | IP | ± | − | − | − | − | − | − | − |
| Fibrin | SC | + | + | ± | − | + | − | − | − |
| | IP | + | − | − | − | − | − | − | − |
| Polymorpho | SC | +++ | + | − | − | − | − | − | − |
| nuclear cells | IP | ++ | ± | − | − | − | − | − | − |
| Lymphocytes | SC | ± | ++ | ++ | ± | + | ± | ± | − |
| | IP | ± | + | + | ± | + | ± | ± | − |
| Macrophages | SC | − | ± | + | + | + | ± | ± | ± |
| | IP | − | ± | + | ± | ± | ± | ± | − |
| Necrosis | SC | − | − | − | − | − | − | − | − |
| | IP | − | − | − | − | − | − | − | − |
| Vascularization | SC | ± | +++ | ++ | + | ++ | ± | + | ± |
| | IP | − | ++ | ++ | + | ++ | ± | ± | ± |
| Fibroblasts | SC | ± | +++ | ++ | + | ++ | + | ± | ± |
| | IP | − | ++ | ± | ± | + | ± | ± | ± |
| Capsul thickness | SC | 0 | 0 | 0 | 100 | 0 | 120 | 100 | 80 |
| (μm) | IP | 0 | 0 | 0 | 80 | 0 | 80 | 80 | 60 |

One day after implantation, the inflammatory response in the surrounding tissues was characterized by the typical appearance of polymorpho nuclear cells (PMN) (FIG. 9A). On day 3, the peri-implant tissue response was dominated by activated fibroblasts with intermingling lymphocytes, occasional macrophages and numerous newly formed small blood vessels (FIG. 9B). After the first week, initiation of peri-implant fibrous capsule formation was present, with macrophages being more conspicuous and dominant in the inflammatory infiltrate (FIG. 9C). On day 14, all implants were encapsulated, the capsules becoming thinner with minimal inflammatory cellularity and occasional capillaries transecting the fibrous tissue. The average thickness of the peri-implant capsule was 100 μm (FIG. 9D).

The surrounding tissue response to intraperitoneal FDC implants showed a similar pattern to that observed after subcutaneous implants, including some degree of degradation of the implant substance. However, the intraperitoneal tissue response was milder and more indolent creating a thinner capsule measuring in average of 80 μm (FIG. 9E).

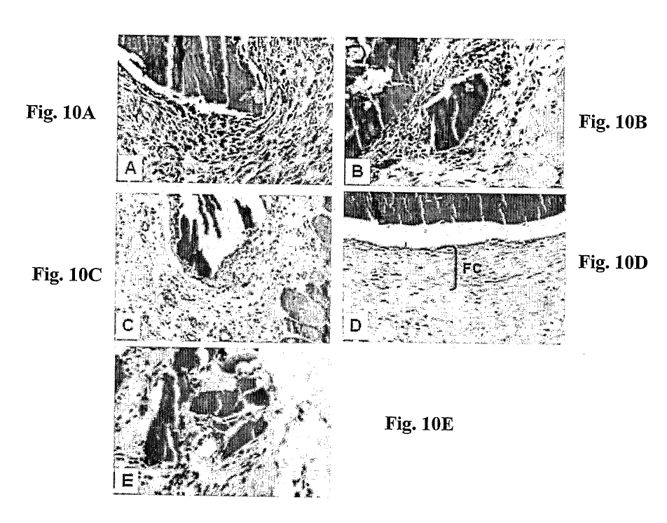

On day 3, the peri-implant tissue response was dominated by activated fibroblasts with mixed cellular response including lymphocytes, macrophages and new vascularization (FIG. 10A). On day 7 post implantation, the peri-implant tissue showed mild mononuclear inflammatory infiltrates, moderate number of activated fibroblasts and newly formed capillaries (FIG. 10B). Within two weeks, both the inflammatory infiltrate and the number of capillaries diminished (FIG. 10C). The activated fibroblasts were gradually replaced by their mature counterparts and a fibrous capsule surrounded the implants. On day 28, the capsule was a cellular with scarce macrophages and few capillaries (FIG. 10D) and the capsule thickness averaged 100 μm.

The surrounding tissue response to intraperitoneal SDC implants showed a similar pattern to that observed following the implantation of subcutaneous implants. However, the intraperitoneal tissue response was milder and more indolent creating a thinner capsule measuring in average of 80 μm (FIG. 9E).

FIG. 11 depicts the adjacent tissue response to SDC after 28 days and FDC after 14 days of subcutaneous implantation, compared to that elicited by a polyglycolic-polylactic absorbable suture after 28 days of subcutaneous implantation. The typical chronic foreign body reaction to biodegradable surgical sutures was not evoked by either gel.

### Example 10

### Toxicity Analysis in Distant Organs

In separate studies three doses (1, 5 and 15 g/kg) of SDC gel and 2 cm of 3/0 polyglycolic-polylactic absorbable suture (Vicryl®, Ethicon, Piscataway, N.J., USA) were implanted SC in the back of four groups of rats for the purpose of pathology comparison to a non-treated group (n=15 rats in each group). Five rats from each group were sacrificed at 4, 14 and 30 days and specimens were taken from brain, lung, kidney, liver, spleen and sternal bone marrow for histological assessment of possible tissue injury and presence of microscopic debris of the implanted objects.

Neither the presence of gel fragments nor tissue damage could be observed in the brain, heart, lung, kidney, liver, spleen and sternal bone marrow of the tested rats at all time points (0, 4, 14 and 30 days), after either implantation of the three doses of the gel (1, 5 and 15 g/Kg), or the polyglycolic-polylactic absorbable suture material.

### Example 11

### Oxidative Degradation of the Gel in-Vitro

Cubic FDC specimens (s=4 mm) were incubated in different concentrations (0, 1, 5 and 10 mM) of KMnO₄ for 3 min. The gels were retrieved, washed twice with water, and incubated separately in 1 ml of hematoxylin (0.05 mg/ml solution) or eosin (0.5 mg/ml solution) for 4 h at room temperature. The concentration of the remaining dye in the incubation medium was measured at 560 nm (hematoxylin) and 520 nm (eosin) and the fraction (percent from initial amount) of dye adsorbed onto the gels was calculated.

FDC gels implanted intraperitonealy in the rat showed signs of partial and total degradation after 7 and 14 days, respectively (FIGS. 12A and B). Degradation changed the eosinophilic gel into a basophilic granular material, some of which was found ingested by the macrophages in the vicinity of the lymph nodes (FIG. 12C).

FIG. 13 shows that the in vitro oxidation of the FDC gel by KMnO₄ caused a decrease in the eosin staining, and an increase in the hematoxylin staining. The amount of stain absorbed was directly correlated to the extent of oxidation.

### Example 12

### Adjacent Tissue Response to Implants Loaded with ¹³¹I-NC

Specimens (0.5 g) of the SDC hydrogels loaded with ¹³¹I-NC (see above) were implanted in the left pectoral region of three anesthetized rat, and the tissue response in the peri-implant tissue was evaluated after 30 days, as described above.

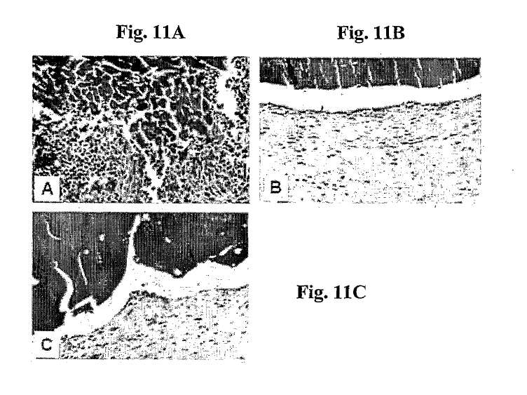

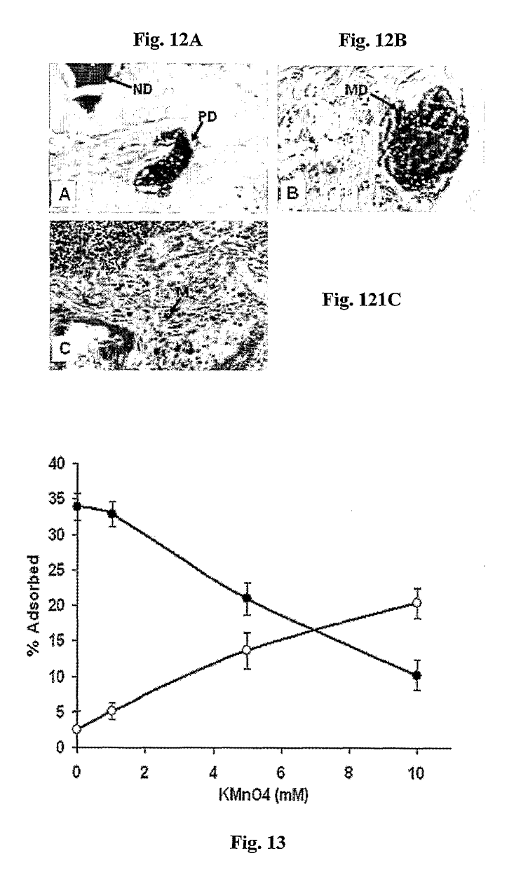

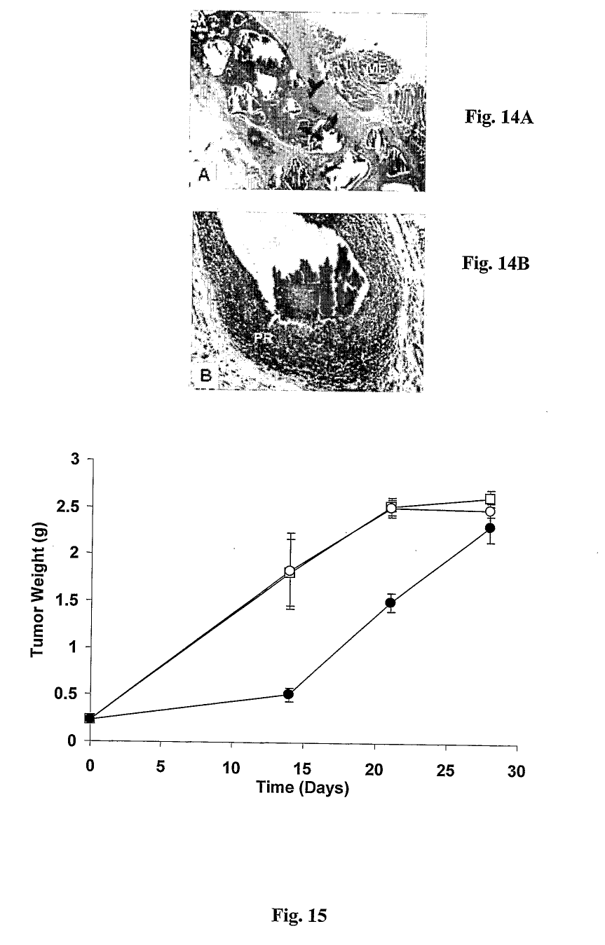

In contrast to the mild tissue response to the non-radioactive SDC and FDC implants, SDC loaded with ¹³¹I-NC caused a profound inflammation in the tissues surrounding the radioactive gel. In some cases liquefactive necrosis was observed (FIGS. 14A and 14B). In all cases, none of the adjacent muscle fibers showed signs of either necrosis or tissue damage.

B—Tumor Recurrence Prevention by Brachytherapy Using Biodegradable Crosslinked Chitosan Hydrogel Implants Loaded with 131I-Nor-Cholesterol

4T1 cells, from metastatic mouse breast cancer, were cultured at 37° C. in a humidified atmosphere of 5% CO₂/air in Dulbecco's Modified Eagle's Medium supplemented with 10% heat-inactivated fetal bovine serum, penicillin G (60 mg/liter), and streptomycin (100 mg/liter). Cells were harvested with Trypsin-EDTA, washed with PBS, and concentrated to 2.5×10⁵ and 2.5×10³ cells/ml in PBS for tumor progression and micro-residual disease studies, respectively.

Female, 7-9 weeks, BALB/c Mice were used in this study, which was conducted in accord with the Principles of Laboratory Animal Care (NIH Publication #85-23, 1985 Revision). Anesthesia was performed by an intraperitoneal injection of 100 mg/kg body weight of ketamine (Ketase™, 0.1 g/ml Fort Dodge, USA). Euthanasia of the anesthetized rats was carried out by chest wall puncturing.

### Example 13

### The Effect of ¹³¹I-Nc Loaded Hydrogels on Tumor Progression

A suspension of 4T1 cells (0.2 ml) was subcutaneously injected in the back of sixty female BALB/c mice (5×10⁵ cells/mouse). Mice were observed for a further two weeks for tumor progression. Mice were anesthetized, a 1-cm incision was performed in the back skin adjacent to the tumor, hydrogels were implanted in the vicinity of the tumor and the skin was sutured. The study was divided into three groups, each group contained 20 mice:

group-1 was a non-treated control group in which no hydrogels were implanted;

group-2 was implanted with 0.5 g of empty hydrogel as a vehicle control, and

group-3 was implanted with 0.5 g of ¹³¹I-NC loaded hydrogels.

Three mice were sacrificed from each group at 2, 3 and 4 weeks. Tumor and internal organs (lung, heart, liver and kidney) were dissected, weighed and analyzed histologically. Moreover, Kaplan-Meier survival analysis was performed over 6 weeks.

Tumor progression rate in untreated and empty-hydrogels treated groups was 0.11 g/day in the first 21 days after the beginning of the treatment, and no significant progression was detected beyond this time (FIG. 15). The tumor progression rate in the group treated with ¹³¹I-NC loaded hydrogels was 0.02 g/day during the first 14 days and 0.12 g/day during days 15 to 28, and no significant progression was detected beyond day 28 (FIG. 15). FIG. 16 depicts the decreased tumor progression (A-C), and the decreased number of metastatic nodes (D-F) in the group treated with ¹³¹I-NC loaded hydrogels as compared to the other two groups 14 days after hydrogel implantation.

In the untreated group and empty-hydrogel treated group mortality initiated at day 17 and completed by day 35 after hydrogel implantation (FIG. 17). On the other hand, in the group treated with 131I-NC loaded hydrogels mortality initiated at day 26 and completed by day 42 after hydrogel implantation (FIG. 17).

### Example 14

### The Effect of ¹³¹I-NC Loaded Hydrogels on Preventing Tumor Recurrence

Tumor cells (10% of the amount utilized for primary tumor implantation) were spread during the implantation of hydrogels in healthy mice, mimicking micro-residual disease in the tumor bed after surgical tumor removal, and tumor cell spillage during the surgical procedure. Sixty mice were anesthetized, a 1-cm incision was performed in the back skin of the mice, hydrogels were implanted, 0.2 ml of cell suspension (5×10³ cells/mouse) were spread dropwise in the implantation site and the skin was sutured. The study was divided into three groups as described above. Histological analysis of organs (lung, heart, liver, kidney and tissue at the site of cell implantation) was performed at 11 weeks after cell injection, and Kaplan-Meier survival analysis was performed over 20 weeks.

All the animals in the untreated group and empty-hydrogel treated group developed tumors and died after 77 and 84 days after hydrogel implantation, respectively. Only 31% of the group treated with ¹³¹I-NC loaded hydrogels developed tumors and died 77 after hydrogel implantation. However, 69% of this group did not develop any tumors and continued to survive until the study was stopped after 160 days (FIG. 18). Tumors developed in all groups during the sixth and seventh week of the micro-residual disease experiments (FIG. 18). FIG. 19 depicts the tumor progression in the untreated and the empty hydrogel treated hydrogel groups and the prevention of tumor recurrence in the group treated with ¹³¹I-NC loaded hydrogels, at week 10.

### Example 15

### Histological Analysis of Adjacent Tissues and Distant Organs

Specimens from tumor bed and distant organs (lungs, heart, liver spleen) were dissected from mice at 14 days for the tumor progression model and 80 days for the tumor recurrence model. Specimens were then rinsed with PBS, fixated with 4% formaldehyde in PBS, dehydrated, embedded in paraffin blocks, sectioned (4 μm) and stained with hematoxylin-eosin for histological examination of tumor progression or recurrence and metastasis in distant organs (FIG. 20).

### Example 16

### Imaging and Estimation of Biological Elimination of the Hydrogel

Specimens (0.5 g) of ¹³¹I-NC loaded hydrogels were implanted subcutaneously in the back of four mice. Scintigraphy was performed at 0, 4, 14 and 30 days after implantation. Each mouse was imaged for 10 min under anesthesia, using a helix dual-head camera (Elscint, Haifa, Israel) and a high-energy, high-resolution collimator. Data was analyzed on a Xeleris program (GE Healthcare), regions of interest were drawn on each focus, and the total number of counts in each region was obtained.

Data from the imaging experiments was expressed as natural logarithm of the fraction of the radioactivity remaining with time after implantation, depicted as open circles in FIG. 21. The amount of radioactivity (Q) at any time (t) after implantation of an initial amount (Q₀), when (λ) is the elimination constant, is given by Eq. 5

Eq. %: Q=Q₀e^{-λt},

which was developed to Eq. 6: −Ln(Q/Q₀)=λt.

Linear regression was performed on these results to obtain the elimination constant (λ=0.136 Day⁻¹). The elimination constant (λ) is composed of the radioactive decay constant (λ_{R}) and the biological elimination constant of the isotope (λ_{B}) as described in Eq. 7. The discontinuous line in FIG. 21, depicts the theoretical radioactive decay of the isotope without considering the biological elimination, accordingly, the radioactive decay constant was λ_{R}=0.0865 Day⁻¹. The biological elimination constant was obtained according to Eq. 7: λ=λ_{R}+λ_{B} (wherein λ_{B}=0.0495 Day⁻¹), and the biological elimination half-life was calculated according to Eq. 8: T_{B1/2}=Ln(2)/λ_{B} (with T_{B1/2}=14.0 Days).

1. A therapeutic source comprising a composite of a polymeric matrix embedded with at least one hydrophobic organic compound selected from cholesterol and norcholesterol, each independently being associated with at least one radioactive atom, wherein said at least one hydrophobic organic compound associated with at least one radioactive atom is substantially non-leachable from said matrix.

2. The therapeutic source according to claim 1, wherein said at least one radioactive atom is selected from: (a) gamma emitting radio-isotopes; (b) beta emitting radio-isotopes; or (c) a combination of a gamma emitting radio-isotope and a beta emitting radio-isotope.

3. The therapeutic source according to claim 1, wherein said at least one radioactive atom is selected from radioactive halogens.

4. The therapeutic source according to claim 3, wherein said at least one radioactive halogen is chemically bonded to said at least one hydrophobic organic compound through a covalent or ionic bond.

5. The therapeutic source according to claim 4, wherein said at least one radioactive halogen is one of iodine or fluorine.

6. The therapeutic source according to claim 5, wherein the radioactive iodine and fluorine are selected from ¹²⁴I, ¹²⁵I, ¹²⁷I, ¹³¹I, and ¹⁸F.

7. The therapeutic source according to claim 1, wherein at least one hydrophobic organic compound associated with at least one radioactive atom is iodo-norcholesterol or iodo-cholesterol.

8. The therapeutic source according to claim 7, wherein said iodo-norcholesterol or iodo-cholesterol is ¹³¹I-norcholesterol and ¹³¹I-cholesterol.

9. The therapeutic source according to claim 1, wherein said at least one radioactive atom is selected from: ¹²⁴I, ¹²⁵I, ¹²⁷I, ¹³¹I, ¹⁸F, ⁹⁰Y, ¹⁶⁶Ho, ¹⁸⁶Re, ¹⁸⁸Re, ⁹⁰Sr, ²²⁶Ra, ¹³⁷CS, ⁶⁰Co, ¹⁹²Ir, ¹⁰³Pd, ¹⁹⁸Au, ⁹⁹Tc, ²⁰¹Th, ⁶⁷Ga, ¹¹¹In and ¹⁰⁶Ru, through a chemical bonding selected from ionic bonding, coordination bonding and intermolecular bonding.

10. The therapeutic source according to claim 1, wherein said polymeric matrix is biocompatible and biodegradable.

11. The therapeutic source according to claim 1, wherein said polymer is a hydrogel.

12. The therapeutic source according to claim 1, wherein said polymeric matrix is a polysaccharide.

13. The therapeutic source according to claim 12, wherein said polysaccharide is selected from natural, synthetic or semisynthetic polysaccharides.

14. The therapeutic source according to claim 12, wherein said polysaccharide is selected from alginic acid, amylopectin, amylose, arabinoxylan, chitosan, chondroitin, glycogen, guar gum, heparin, hyaluronic acid, insulin, pectin, and xyloglucan.

15. The therapeutic source according to claim 1 suitable for implantation or instillation in the body or onto the skin of a subject in need of radio-therapy.

16. A therapeutic source comprising a composite of chitosan and at least one radioactive lipid selected from cholesterol and norcholesterol.

17. A method for the preparation of a therapeutic source according to claim 1, said method comprising:
(a) admixing at least one polymer and at least one hydrophobic organic compound selected from cholesterol and norcholesterol, being independently associated with at least one radioactive atom in water or an aqueous solution; and
(b) affecting gelation or hydrogelation of the mixture of step (a),
thereby obtaining a composite of a polymeric matrix embedded with at least one hydrophobic organic compound selected from cholesterol and norcholesterol, being independently associated with at least one radioactive atom, said at least one hydrophobic organic compound associated with said at least one radioactive atom being substantially non-leachable from said matrix.

18. The method according to claim 17, wherein said polymer is a polysaccharide.

19. The method according to claim 17, further comprising the step of washing or incubating the composite in a suitable media.

20. A therapeutic source obtained by the method of claim 17.

21. The therapeutic source according to claim 1, suitable as radioactive medical implant.

22. The therapeutic source according to claim 1, suitable for internal local radiation therapy (brachytherapy).

23. The therapeutic source according to claim 1, suitable for injection directly into a tumor.

24. A radioactive medical implant for placement at a surgical site or a body cavity comprising a therapeutic source according to claim 1.

25. The radioactive medical implant according to claim 24, wherein said surgical site or a body cavity is a site of a surgically removed tumor.

26. Use of a composite for the preparation of a therapeutic source, said composite being of a polymeric matrix embedded with at least one hydrophobic organic compound selected from cholesterol and norcholesterol, each independently being associated with at least one radioactive atom, wherein said at least one hydrophobic organic compound associated with at least one radioactive atom is substantially non-leachable from said matrix.

27. The use according to claim 26, wherein said polymeric matrix being biocompatible and biodegradable.

28. The use according to claim 27, wherein said therapeutic source being suitable for implantation or instillation in the body or onto the skin of a subject in need of radio-therapy.
